(19) United States
(12) Patent Application Publication
(10) Pub. No.: US 2012/0191013 A1
(43) Pub. Date: Jul. 26, 2012

(54) System to select condom using hand held electronic measuring device

(76) Inventors: Samuel J. Nelson, Liantarnam Cwmbran Torfean (GB); Joseph R. Nelson, London (GB)

(21) Appl. No.: 13/374,826

(22) Filed: Jan. 17, 2012

Related U.S. Application Data
(60) Provisional application No. 61/461,450, filed on Jan. 18, 2011.

(51) Int. Cl. A61B 5/103

(52) U.S. Cl. 600/587

(57) ABSTRACT

A method for fitting a wearable covering for a body part comprises the steps of running a program on a portable terminal; determining with the portable terminal specific geometrical points regarding the body part; using the determined specific geometrical points to determine an approximate shape of the body part; comparing the determined approximate shape with a table of different sized wearable coverings for the body part; and selecting a size for the wearable covering.

This application claims priority based on U.S. Provisional Patent Application Ser. No. 61/461,450 filed Jan. 18, 2011.

This invention relates to condoms.

More particularly, the invention relates to a method and apparatus to operatively associate a hand held computer, an individual's erect penis, and the Internet to define and provide a customized condom for the individual.

A long existing motivation in the field of sexual intimacy is to provide a properly fitting condom to reduce both the transmission of venereal disease and the incidence of unintended pregnancies.

Accordingly, it would be highly desirable to provide an improved method and apparatus to provide an individual a properly fitting condom.

Therefore, it is a principal object of the invention to provide an improved method and apparatus for an individual to use to select a condom.

This and other, further and more specific objects of the invention will be apparent to those skilled in the art from the following detailed description thereof, taken in conjunction with the drawings, in which:

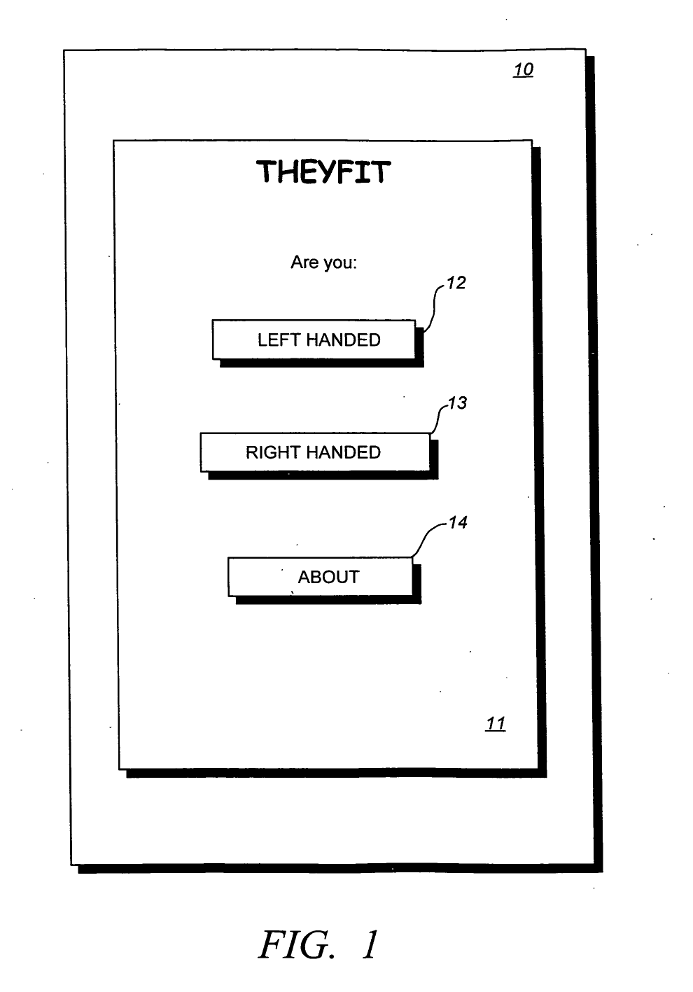

FIG. 1 is a front view of a digital audio player (DAP) constructed in accordance with the invention and displaying the first page of a specialized computer program constructed in accordance with the invention and incorporated in and utilized in combination with the digital audio player and an individual's erect penis to design for the individual a customized condom;

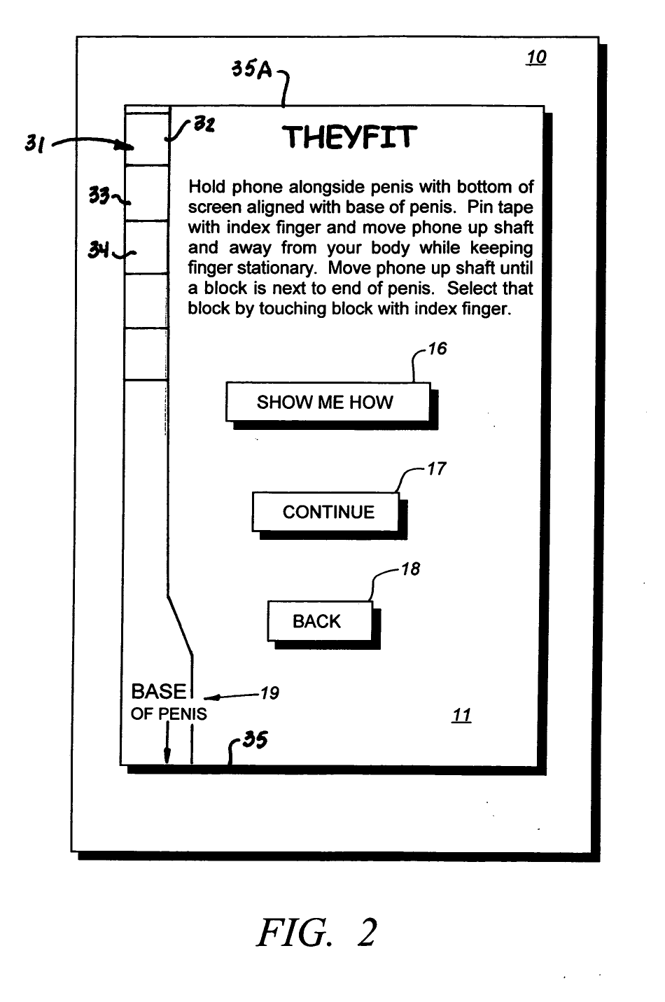

FIG. 2 is a front view of the DAP of FIG. 1 displaying the second page of the specialized computer program;

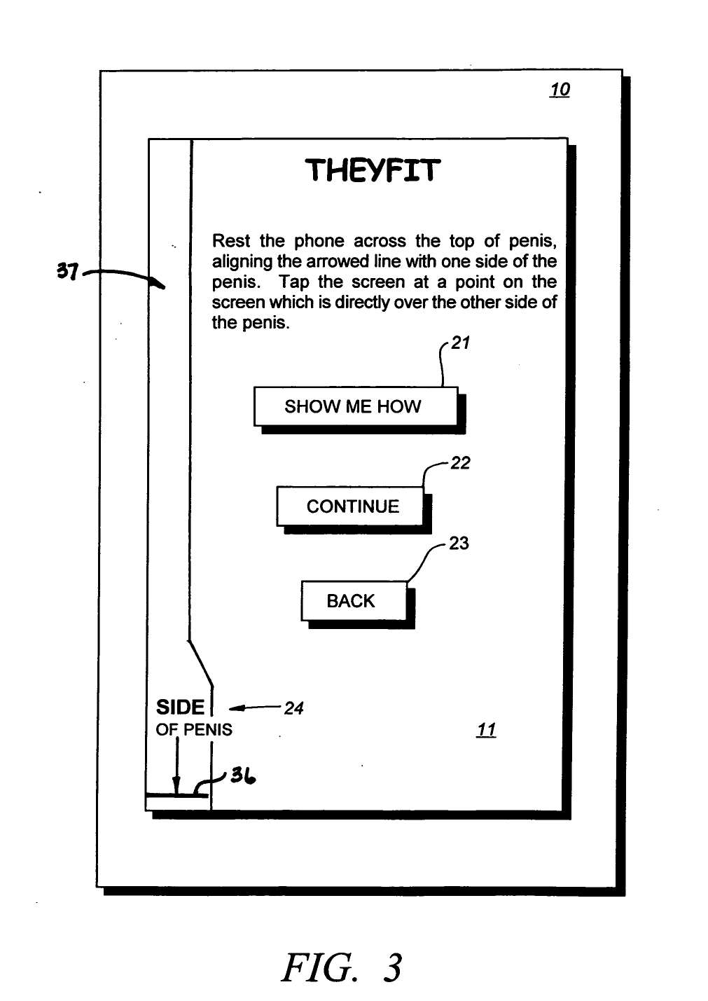

FIG. 3 is a front view of the DAP of FIG. 1 displaying the third page of the specialized computer program;

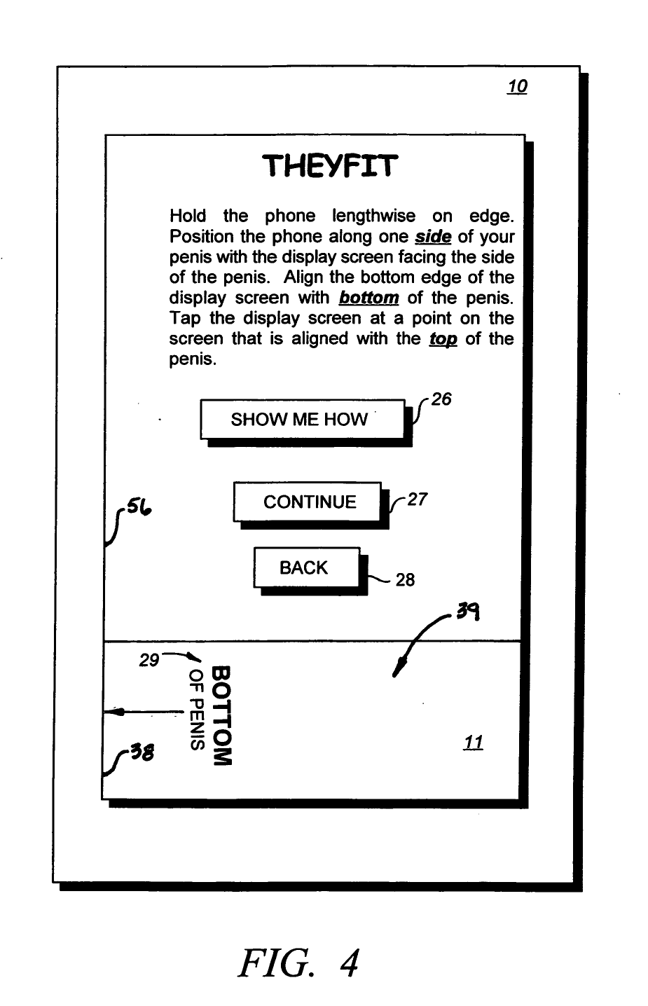

FIG. 4 is a front view of the DAP of FIG. 1 displaying the fourth page of the specialized computer program;

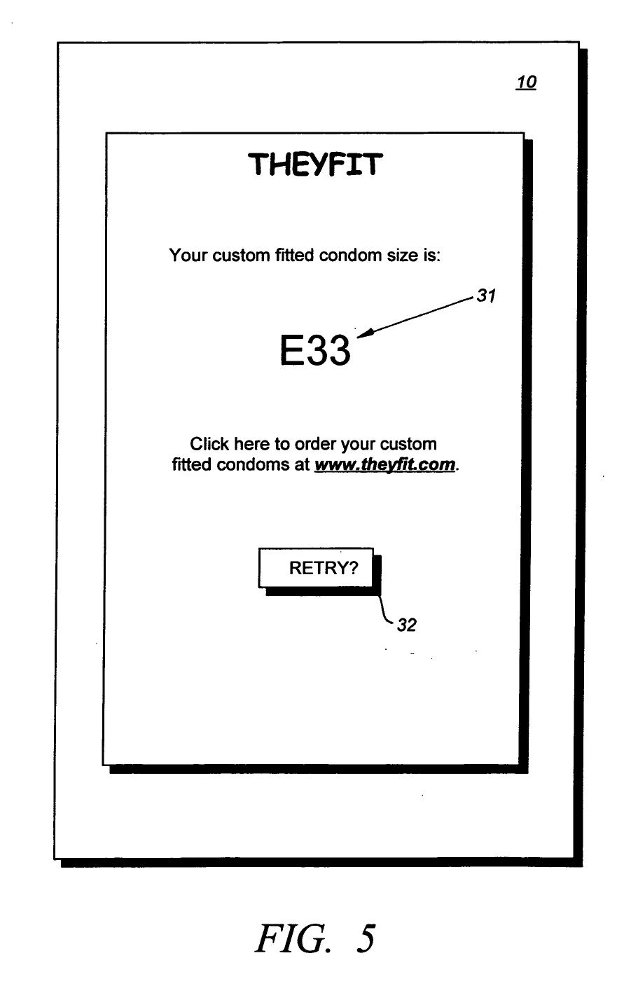

FIG. 5 is a front view of the DAP of FIG. 1 displaying the fifth page of the specialized computer program;

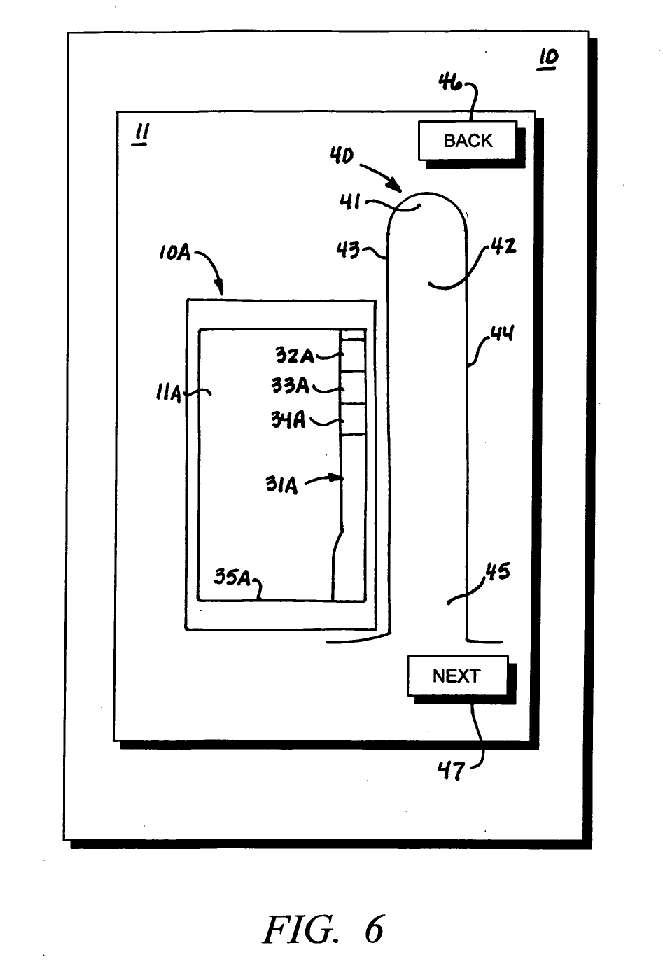

FIG. 6 is a front view of the DAP of FIG. 1 displaying the primary instruction page used in conjunction with the second page shown in FIG. 2 and illustrating the mode of operation of said second page;

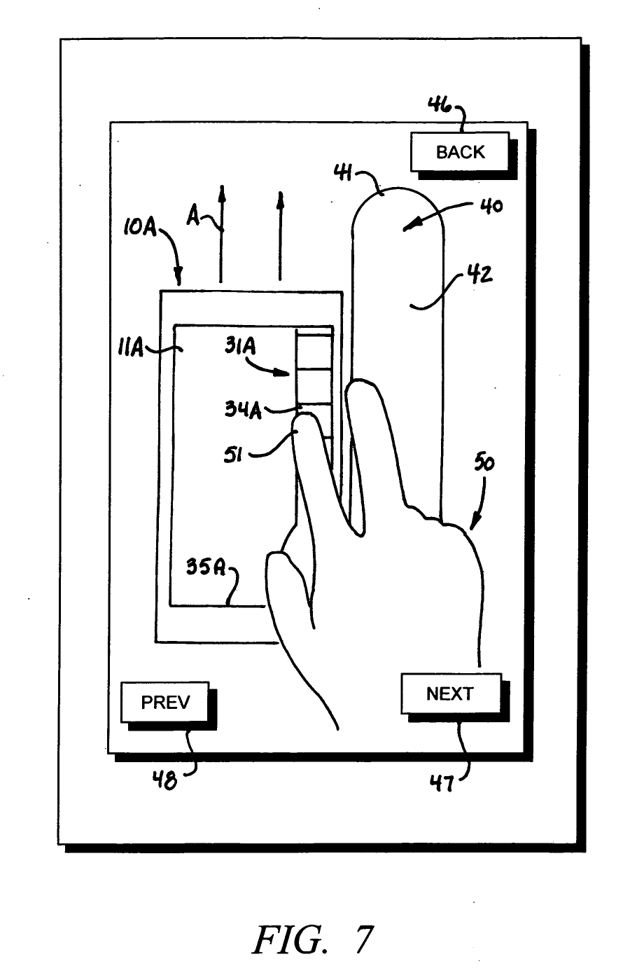

FIG. 7 is a front view of the DAP of FIG. 1 displaying the secondary instruction page used in conjunction with the second page shown in FIG. 2 and further illustrating the mode of operation of said second page;

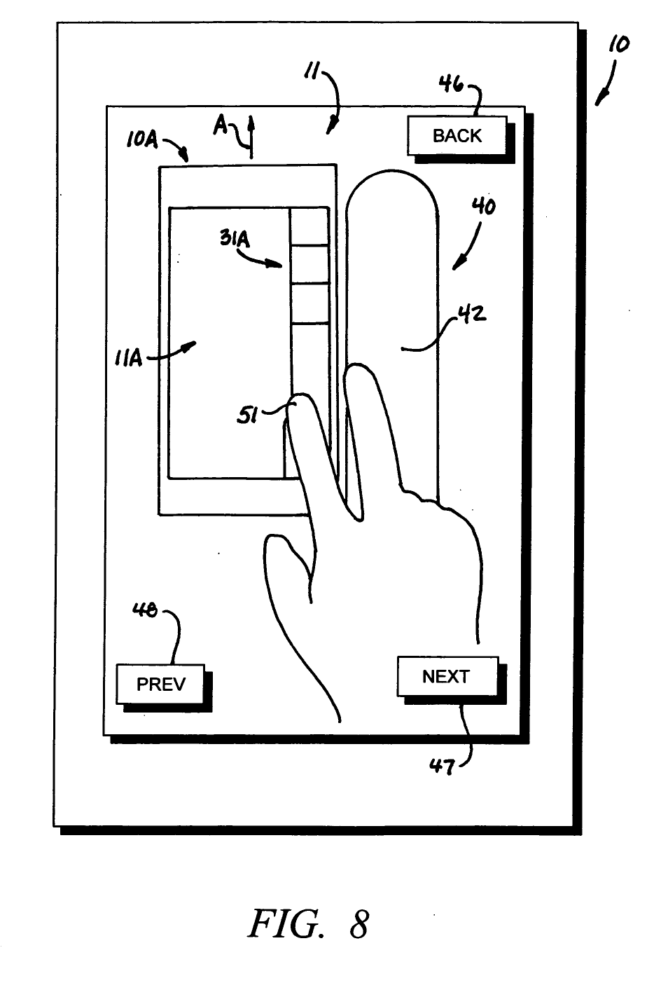

FIG. 8 is a front view of the DAP of FIG. 1 displaying the tertiary instruction page used in conjunction with the second page shown in FIG. 2 and illustrating still further the mode of operation of said second page;

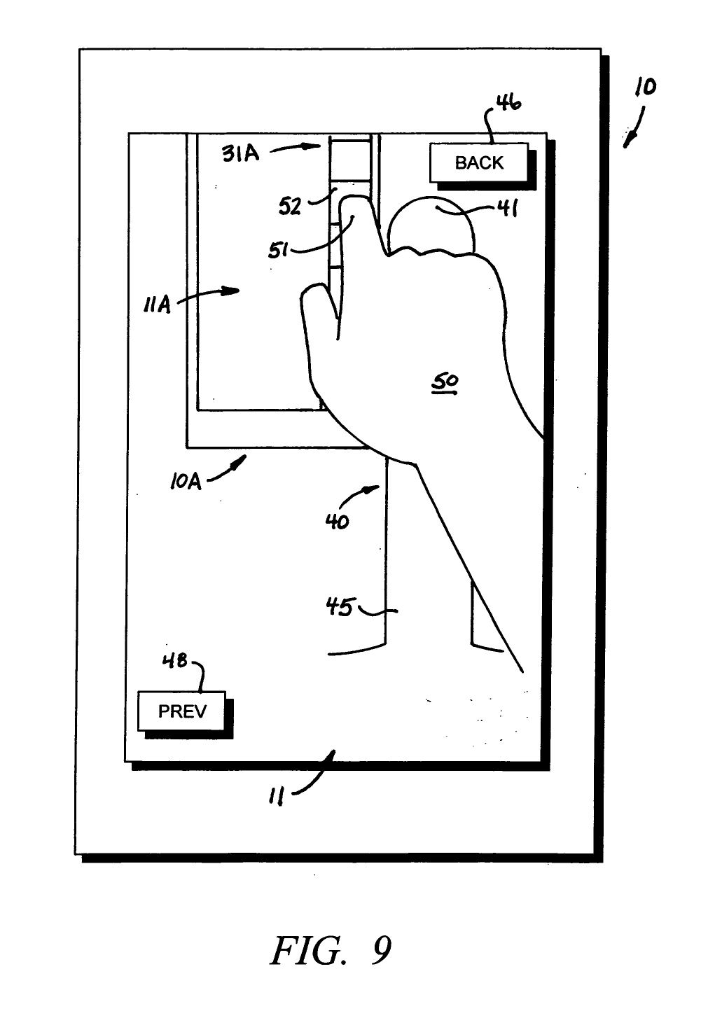

FIG. 9 is a front view of the DAP of FIG. 1 displaying the quaternary instruction page used in conjunction with the second page shown in FIG. 2 and illustrating yet further the mode of operation of said second page;

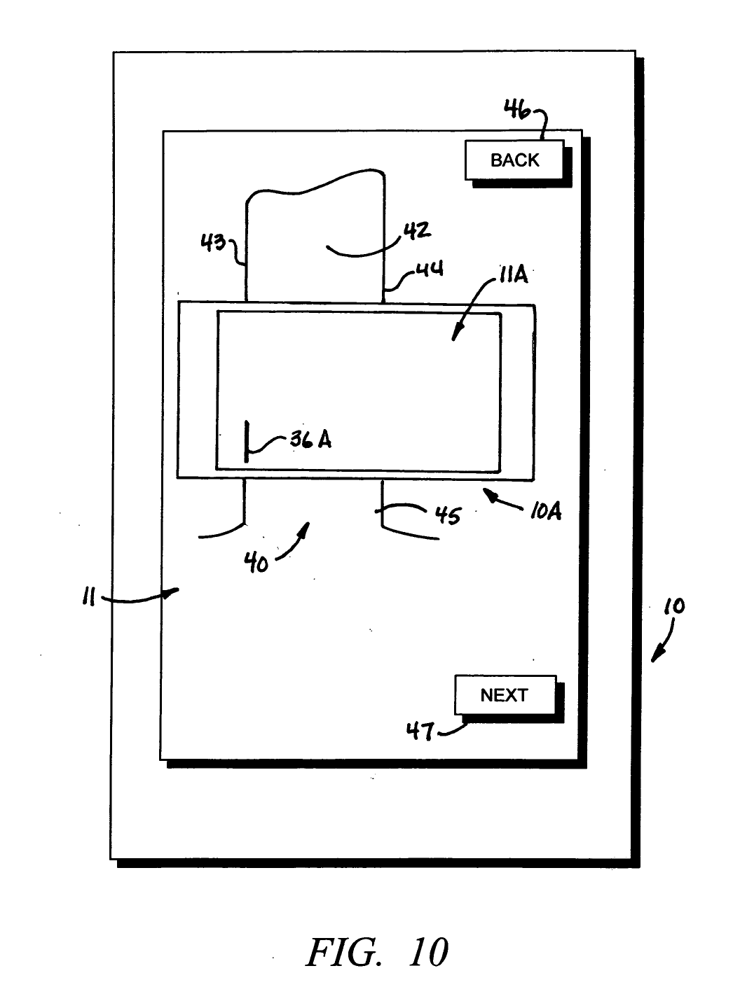

FIG. 10 is a front view of the DAP of FIG. 1 displaying the 1st instruction page used in conjunction with the third page shown in FIG. 3 and illustrating the mode of operation of said second page;

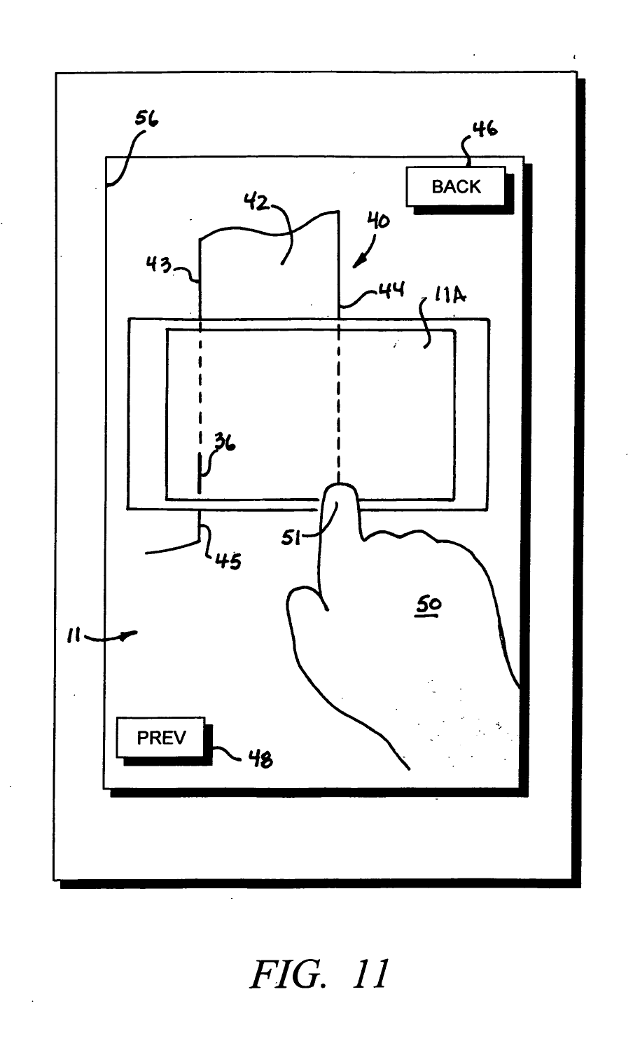

FIG. 11 is a front view of the DAP of FIG. 1 displaying the 2nd instruction page used in conjunction with the third page shown in FIG. 3 and further illustrating the mode of operation of said third page;

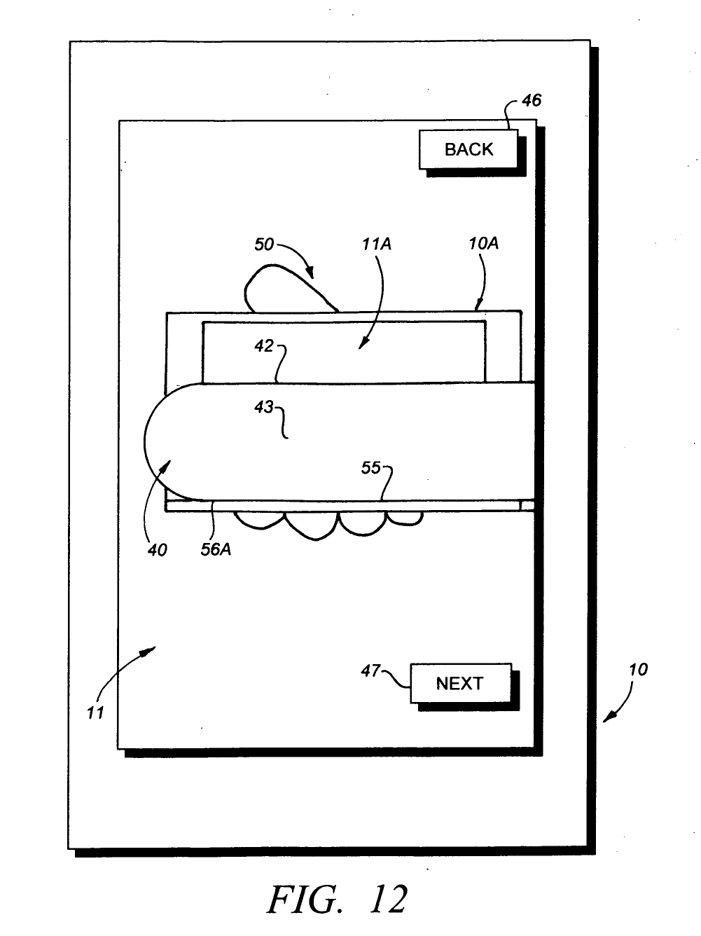

FIG. 12 is a front view of the DAP of FIG. 1 displaying the preliminary instruction page used in conjunction with the fourth page illustrated in FIG. 4 and illustrating the mode of operation of said fourth page;

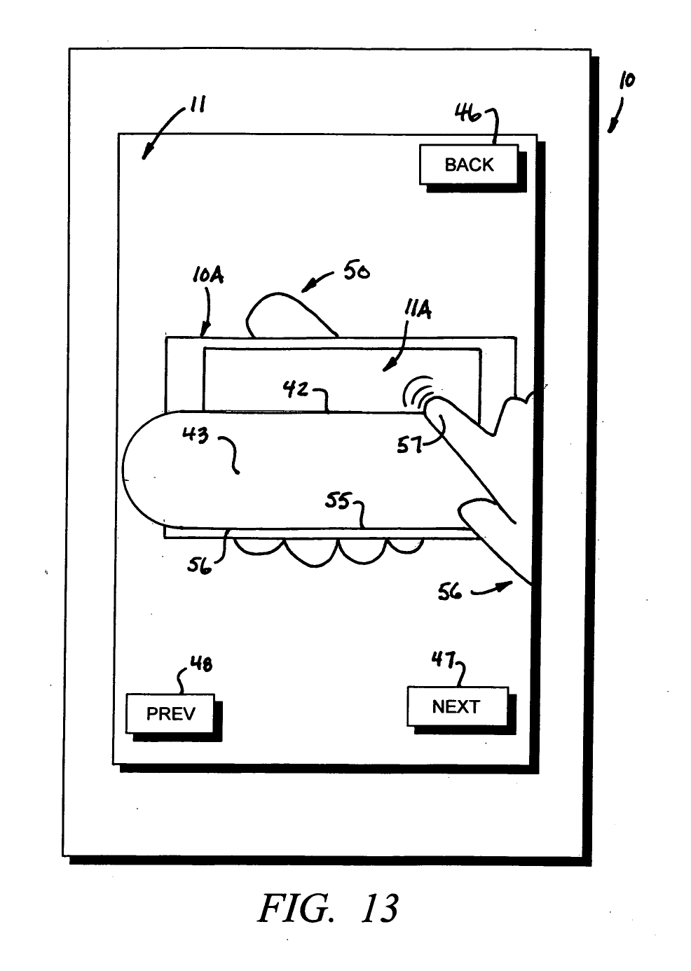

FIG. 13 is a front view of the DAP of FIG. 1 displaying the secondary instruction page used in conjunction with the fourth page illustrated in FIG. 4 and further illustrating the mode of operation of said fourth page;

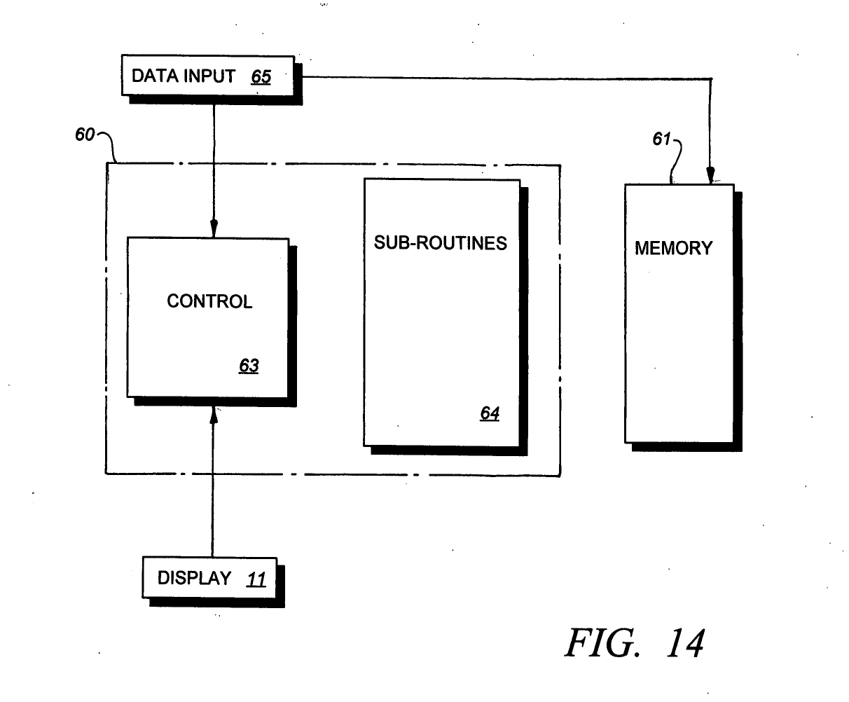

FIG. 14 is a block diagram illustrating the computer system utilized in the DAP of FIG. 1;

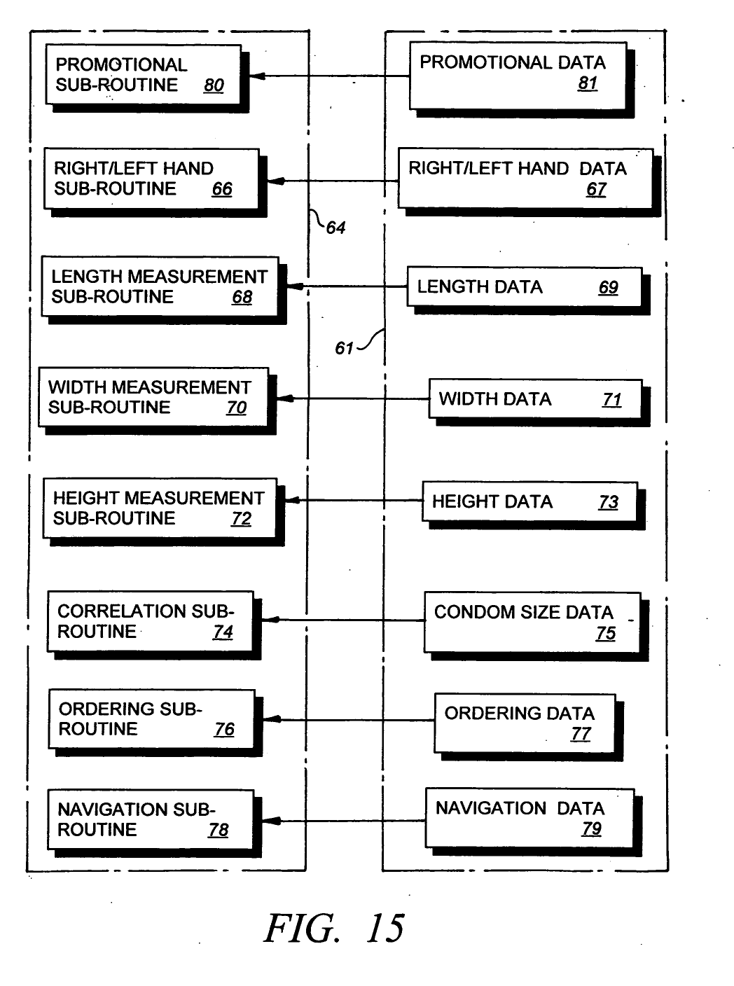

FIG. 15 is a block diagram illustrating sub-routines and memory data utilized in the computer program of the computer in FIG. 14;

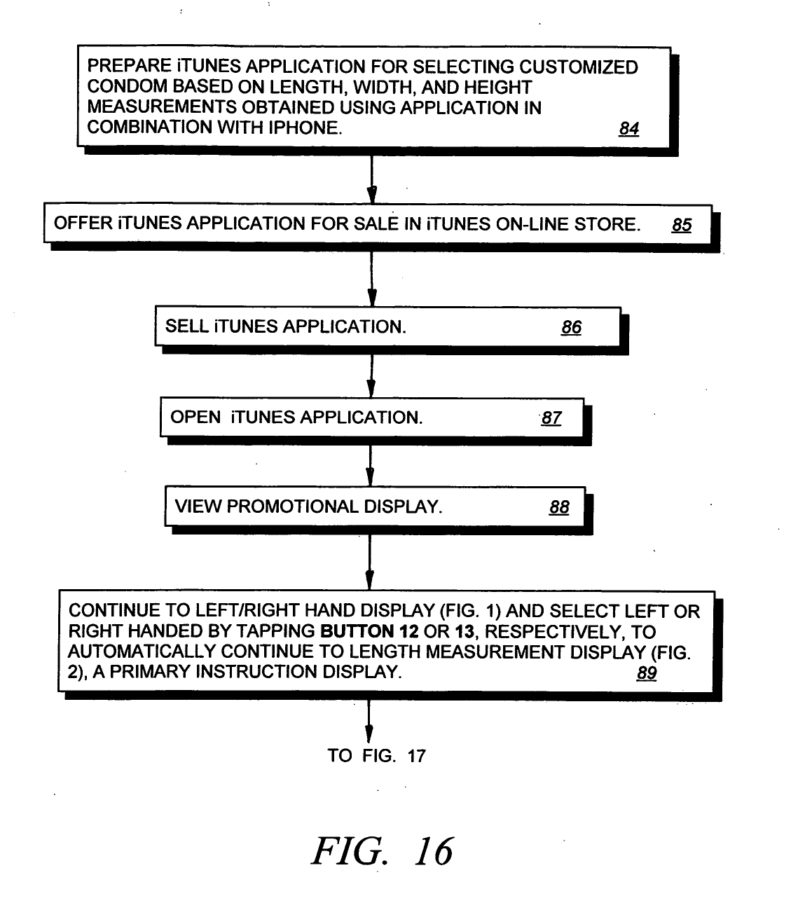

FIG. 16 is a block flow diagram illustrating the use of the DAP of FIG. 1 and of the specialized computer program utilized therein;

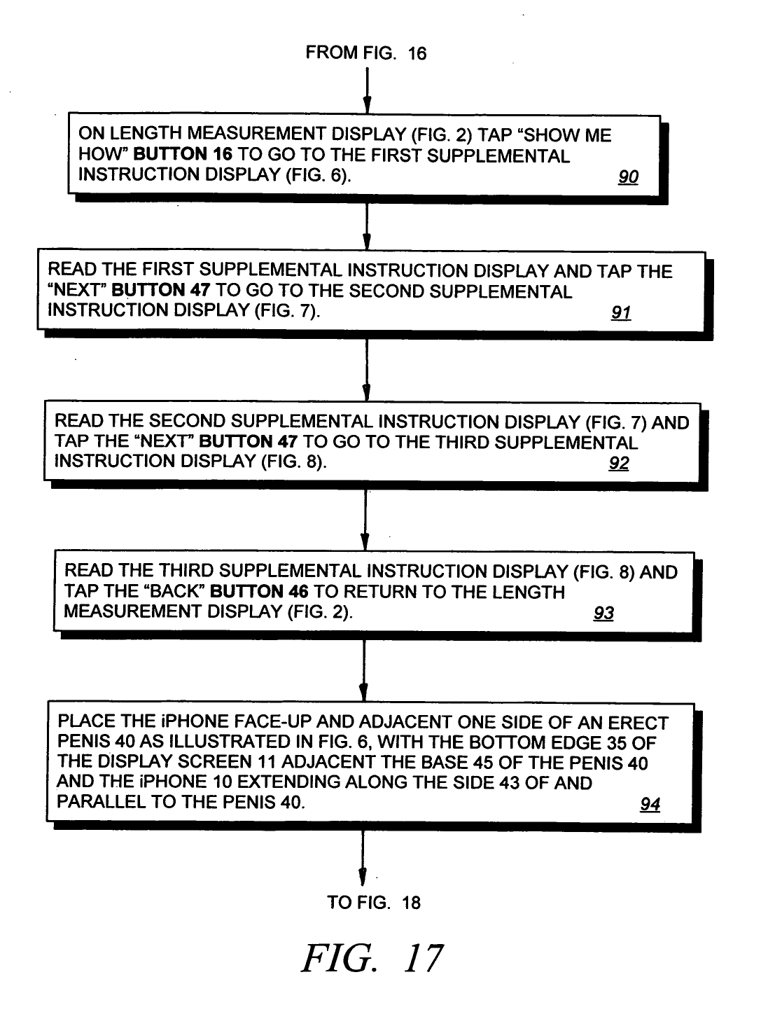

FIG. 17 is a continuation of the block flow diagram of FIG. 16 further illustrating the use of the DAP of FIG. 1;

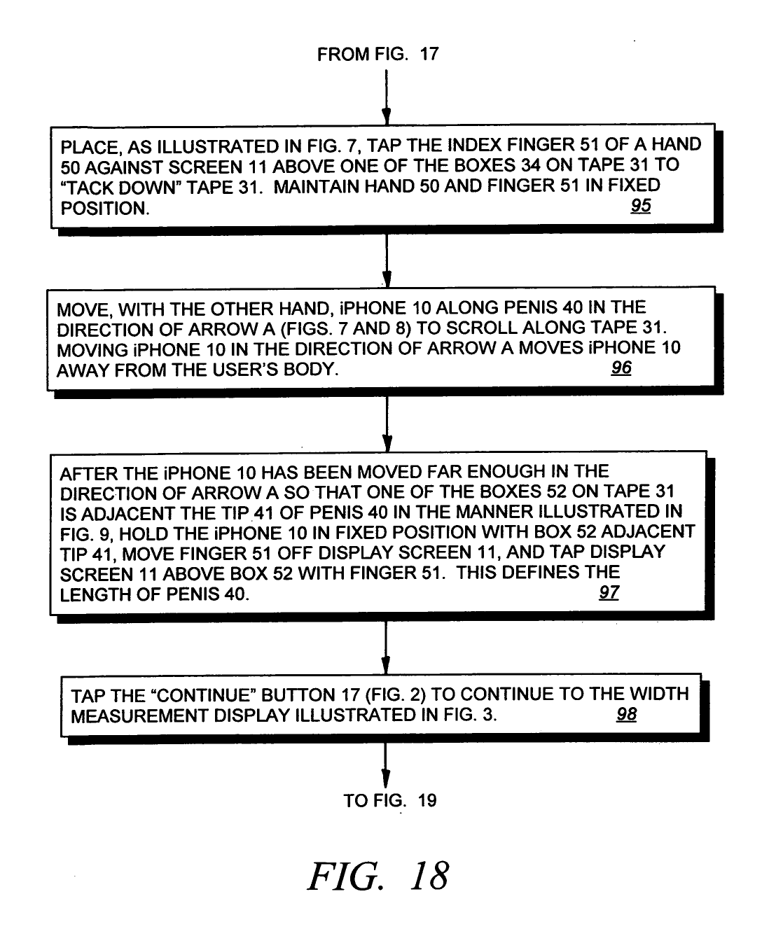

FIG. 18 is a continuation of the block flow diagram of FIG. 17 further illustrating the use of the DAP of FIG. 1;

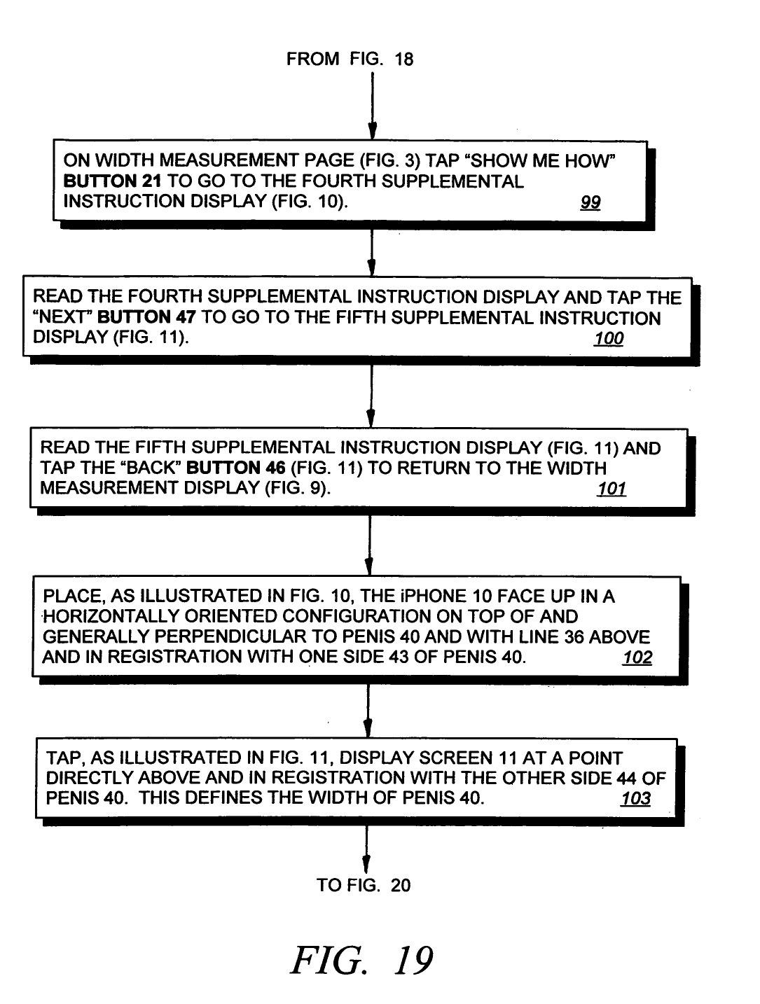

FIG. 19 is a continuation of the block flow diagram of FIG. 18 further illustrating the use of the DAP of FIG. 1;

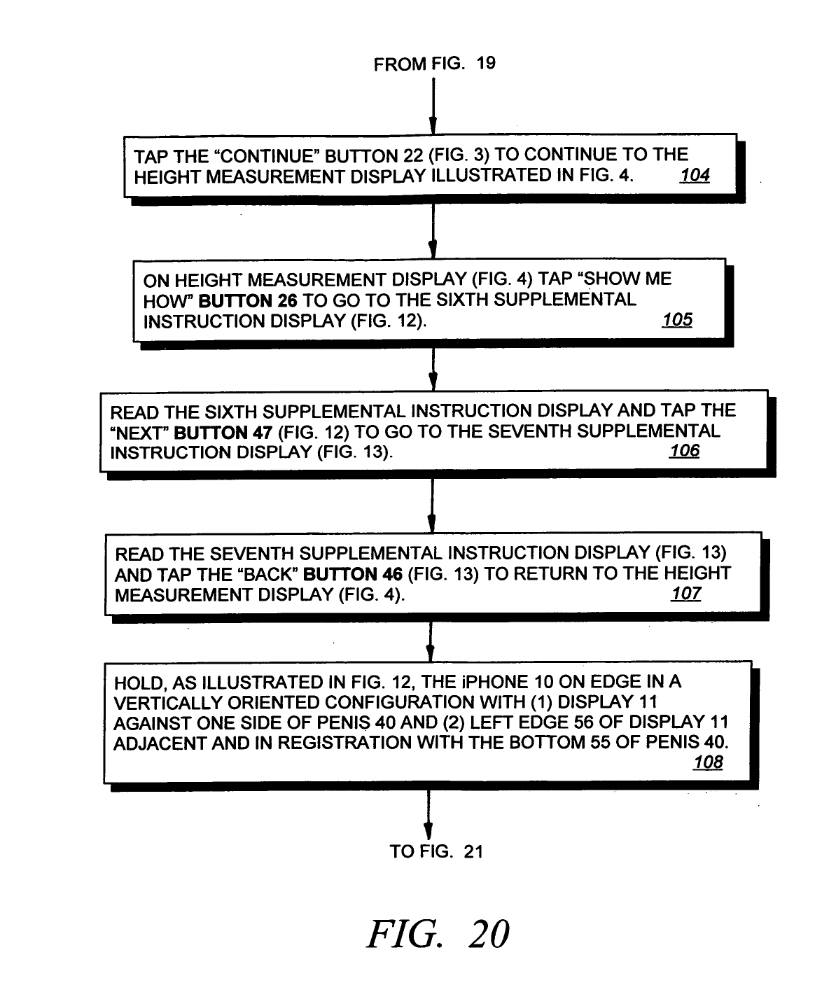

FIG. 20 is a continuation of the block flow diagram of FIG. 19 further illustrating the use of the DAP of FIG. 1; and

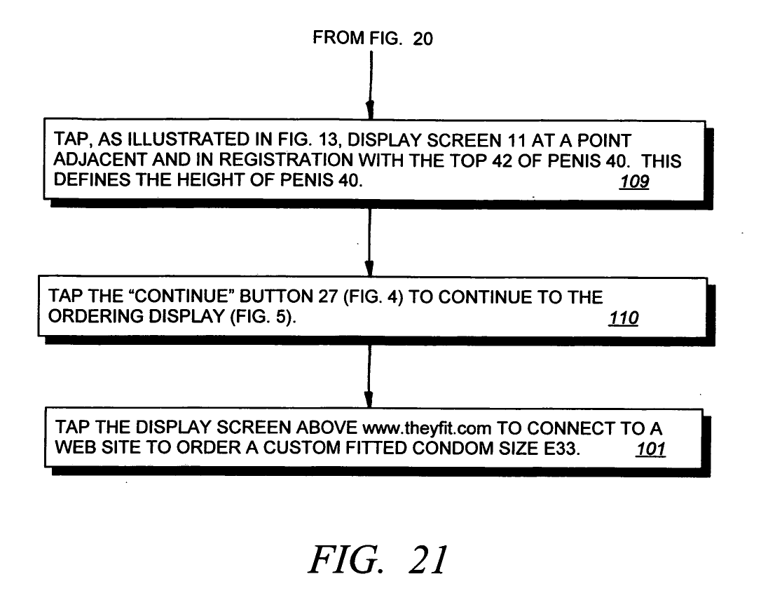

FIG. 21 is a continuation of the block flow diagram of FIG. 20 further illustrating the use of the DAP of FIG. 1.

Briefly, in accordance with the invention, I provide an improved method for fitting a wearable covering for a body part. The method comprises the steps of running a program on a portable terminal; determining with the portable terminal specific geometrical points regarding the body part; using the determined specific geometrical points to determine an approximate shapes of the body part; comparing the determined approximate shape with a table of different sized wearable coverings for the body part; and, selecting a size for the wearable covering.

In another embodiment of the invention, I provide an improved method for an individual to select a condom. The method comprises the step of designing a specialized computer program for use on a hand held computer device. The hand held computer device includes a touch sensitive display screen on which data commands are entered by touching the display screen at selected areas on the screen. The specialized computer program utilizes the touch screen to measure the length, width, and height of an erect penis; utilizes a specialized algorithm to calculate the girth of an erect penis, the penis including a top, a bottom, and a pair of spaced apart generally parallel sides; and, utilizes a chart correlating the length and girth of an erect penis to a particular size of condom. The hand held computer device is held in fixed position adjacent an erect penis during measurement of each dimension in a group of dimensions consisting of the length of the width of an erect penis and the height of an erect penis; and, is held during measurement of the length of an erect penis, initially in fixed position adjacent the erect penis and then being moved to scroll along a measuring tape depicted on the display screen of the computer device. The method also comprises the steps of offering the computer program for sale on the Internet; selling the computer program on the Internet; downloading said computer program over the Internet to said handheld computer device; utilizing the handheld computer device and computer program to measure the length of an erect penis of an individual by initially placing the computer device adjacent the erect penis and then moving the computer device along the erect penis away from the body of the individual to scroll along a measuring tape depicted on the display screen of the computer device; utilizing the handheld computer device and computer program to measure the width of the individual's erect penis while holding the computer device in fixed position on the top or bottom of the penis; utilizing the handheld computer device and computer program to measure the height of the individual's erect penis while holding the computer device in fixed position along one of the sides of the penis; utilizing the width and height and algorithm to determine the girth of the penis; utilizing the length and girth to select a particular size of condom from the chart; and, utilizing an Internet web site to order the particular size of condom.

Turning now to the drawings, which depict the presently preferred embodiments of the invention for the purpose of illustration thereof, and not by way of limitation of the invention, and in which like characters refer to corresponding elements throughout the several views, FIGS. 1 to 13 illustrate, by way of example and not limitation, displays that appear on the screen of a hand held digital audio player (DAP), namely an iPhone™ computer device 10, during the utilization of a computer application constructed in accordance with the principles of the invention. The iPhone is about four and five-eighths inches long by two and three-eighths inches wide. As would be appreciated by those of skill in the art, any hand held computer device which includes a display screen, a computer program, and any other necessary operatively associated hardware, firmware, or other apparatus can be utilized in the practice of the invention. By way of example, and not limitation, an iPod™, Blackberry™, Android™, or iPad™ computer device can be utilized.

After a particular “app” (i.e., computer program) from iTunes or another computer application provider is loaded onto an iPhone, the procedure for selecting on the iPhone the icon for that application and tapping the icon to open the application is well known and is not described herein. In the example set forth in FIGS. 1 to 13, after an application for determining a custom condom size is opening, the initial display that appears on the display screen 11 of iPhone 10 is illustrated in FIG. 1. If desired, the initial display can consist of promotional material which a user can read and then, by tapping on a “CONTINUE” button, sequence to the display illustrated in FIG. 1. The promotional material might comprise, for example:
THEYFIT
Custom fit condoms from TheyFit
A revolution in safer sex. We off 95 custom fit sizes for your enhanced comfort. Custom fitting makes everything tit better, and things that fit better feel better—shoes, shirts, pants. Now TheyFit brings this common sense approach to condoms too!
   If desired, the initial display of promotional material (or any other display in the program) can include a link to a video to provide dynamic, practical instruction on how to use the computer program on the mobile computing device. The video can be included in the computer program on the mobile computing device or can be stored at a web site on the Internet.

### Left/Right Handed

The display of FIG. 1 is utilized to identify whether a user is left or right handed. If a user is left handed he taps touch screen 11 at a point above button 12. The program then sequences to the next display illustrated in FIG. 2. If a user is right handed, he taps screen 11 at a point above button 13. The program then sequences to the next display illustrated in FIG. 2. If the user is interested in background information about the computer program, he taps screen 11 at a point above button 14 and the program sequences to a screen which provides background information. As used below, “tapping a button” to “tap a button” indicates that a user is tapping screen 11 at a point above a button that forms part of a display on screen 11. Since screen 11 is a touch screen, tapping the screen at a point above a button functions to generate a command that is sent to the computer in iPhone 10.

The displays shown in FIGS. 2, 3, 4, 5 are primary instruction displays. The displays shown in FIGS. 5 to 13 are supplemental instruction displays.

The display illustrated in FIG. 2 includes “SHOW ME HOW” button 16, “CONTINUE” button 17, and “BACK” button 18.

Whenever a “BACK” button appears in a display appearing on screen 11, tapping the “BACK” button causes the program to sequence back to the immediately preceding primary instruction display.

Whenever a “CONTINUE” button appears in a display appearing on screen 11, tapping the “CONTINUE” button causes the program to index forward to the next immediately succeeding primary instruction display.

Whenever a “PREV” button appears in a display appearing on screen 11, tapping the “PREV” button causes the program to index back to the next immediately preceding supplemental instruction display.

Whenever a “NEXT” button appears in a display appearing on screen, tapping the “NEXT” button causes the program to index forward to the next immediately succeeding supplemental instruction display.

### Length Determination

A touch-drag (or scroll)-touch measurement methodology is utilized to determine penile length. The iPhone 10 is placed in a “display screen 11 facing up” first orientation generally parallel to and along one side of an erect penis. The first orientation is illustrated in FIGS. 6 to 9.

The primary instruction display of FIG. 2 also includes a tape 31 with marked off incremental blocks 32, 33, 34. A user “pins” the tape 31 by gently pressing his finger against screen 11 above a block 32, 33, 34. After the tape is “pinned” moving iPhone 10 in a direction parallel to tape 31 caused the display screen 11 to scroll along tape 31. In other words, it appears on screen 11 as if tape 31 is in fixed position and that screen 11 is moving along tape 31. The words “BASE OF PENIS” 19 in conjunction with the arrow that is pointing to the lower edge 35 of screen 11, indicating that edge 35 is the edge to be aligned with the base of the user's erect penis.

The display of FIG. 2 includes an instruction paragraph which begins “Hold phone alongside . . . .” After reading the instruction paragraph, the user taps the “SHOW ME HOW” button 16 to index the program to the supplemental instruction display shown in FIG. 6. This supplemental instruction display depicts a penis 40 with a tip 41, top 42, base 45, and opposing, spaced apart sides 43 and 44. The depiction of penis 40 in the display normally comprises a simplified representation of the outline of a penis since a photograph or other accurate rendering of a penis could be characterized as pornographic. The display also depicts an iPhone 10A with a display screen 11A, a lower edge 35A, and with a display depicting a tape 31A having marked off blocks 32A, 33A, 34A. The iPhone 10A and the display on screen 11A correspond to the display that is shown in FIG. 2 and that the user will employ to measure the length of his erect penis. Accordingly, the display of FIG. 6 illustrates how a user positions an iPhone 10A (i.e., the user's iPhone 10) alongside his erect penis with the bottom edge 35A (i.e., the bottom edge 35 of the screen 11 of the user's iPhone 10) of the iPhone aligned with the base 45 of the penis.

Tapping the “BACK” button would sequence the program back to the primary instruction display shown in FIG. 2. Instead, the user taps the “NEXT” button 47 to sequence to the next supplemental instruction display illustrated in FIG. 7.

The display illustrated in FIG. 7 is identical to that of FIG. 6 except that the FIG. 7 display includes (1) a “PREV” button 48, (2) a hand 50 with the tip of the index finger 51 on marked off block 34A, and (3) arrows A indicating how iPhone 10A is moved in the direction of arrows A to slide screen 11 over the tip of finger 51 and scroll screen 11 over tape 31A while tape 31A appears to remain stationary. Hand 50 and finger 51 remain stationary while iPhone 10A is moved in the direction of arrows A.

Tapping the “PREV” button 48 shown in FIG. 7 would sequence the program back to the supplemental instruction display shown in FIG. 6. Tapping the “BACK” button 46 shown in FIG. 7 would sequence the program back to the primary instruction display shown in FIG. 2. Instead, the user taps the “NEXT” button 47 shown in FIG. 7 to sequence to the next supplemental instruction display illustrated in FIG. 8.

The display illustrated in FIG. 8 is identical to that of FIG. 7 except that the FIG. 8 display illustrates how iPhone 10A has continued to be moved along the side of erect penis 40 in the direction of arrow A while the hand 50 and finger 51 have remained in fixed position. On screen 11A, iPhone 10A appears to be scrolling along tape 31A while tape 31A remains in fixed position.

Tapping the “PREV” button 48 shown in FIG. 8 would sequence the program back to the supplemental instruction display shown in FIG. 7. Tapping the “BACK” button 46 shown in FIG. 8 would sequence the program back to the primary instruction display shown in FIG. 2. Instead, the user taps the “NEXT” button 47 shown in FIG. 8 to sequence to the next supplemental instruction display illustrated in FIG. 9.

The display illustrated in FIG. 9 is identical to that shown in FIG. 8 except that (1) the iPhone 10A has been moved further along the side of penis 40 until a marked off block 52 of tape 31A is adjacent the tip 41 of erect penis 40, and (2) the tip of the index finger 51 has lifted off display 11, hand 50 has been moved away from the base 45 of penis 40 toward the tip 41 of penis, and the tip of finger 51 has tapped screen 11A above block 52. Tapping screen 11A above-block 52 determines the length of penis 40.

Tapping the “PREV” button 48 shown in FIG. 9 would sequence the program back to the supplemental instruction display shown in FIG. 8. Instead, the user taps the “BACK” button 46 shown in FIG. 9 to sequence back to the primary instruction display shown in FIG. 2.

The user follows the instructions set forth at the top of the display shown in FIG. 2 to measure the length of his erect penis. The user positions the iPhone 10 face-up adjacent a side of his penis, “pins” the tape 31 with the tip of his index finger 51, and moves the iPhone 10 in the direction of arrow A toward tip 41 while maintaining his hand and finger 51 in fixed position. Once a block 52 on tape 31 is adjacent tip 41, the movement of iPhone 10 is stopped, the user lifts his finger tip off screen 11, and, the user taps block 52 with the tip of his finger to define the length of penis 40. If it is necessary to move iPhone 10 in the direction of arrow A a distance that is greater than the height of screen 11, the movement of iPhone is halted temporarily, finger 51 is lifted from screen 11 and moved toward the top 35A (FIG. 2), and the tip of finger 51 is pressed against a block on tape 31 that is closer to the top of screen 11, and iPhone 10 is then once again moved in the direction of arrow A until a block on tape 31 is adjacent the tip 41 of penis 41.

After the display of FIG. 2 is utilized to determine the length of penis 40, the user taps the “CONTINUE” button 22 (FIG. 2) to sequence to the next primary instruction display that is illustrated in FIG. 3.

### Width Determination

An align-touch measurement methodology is utilized to determine penile width. The iPhone 10 is placed in a “display screen 11 facing up” second orientation generally perpendicular to and transverse an erect penis. The second orientation is illustrated in FIGS. 10 and 11.

The primary instruction display illustrated in FIG. 3 includes “SHOW ME HOW” button 21, “CONTINUE” button 22, and “BACK” button 23.

The primary instruction display of FIG. 3 also includes the words “SIDE OF PENIS” 24 in conjunction with an arrow that is pointing to the line 36, indicating that line 36 is to be aligned with a side of the user's erect penis.

The display of FIG. 3 includes at the top of the display an instruction paragraph which begins “Rest the phone . . . .” After reading the instruction paragraph, the user taps the “SHOW ME HOW” button 21 to index the program to the supplemental instruction display shown in FIG. 10. This supplemental instruction display depicts the proximate portion of penis 40 including top 42, sides 43 and 44, and base 45. The display also depicts an iPhone 10A with a display screen 11A, and a line 36A depicted on screen 11A. The iPhone 10A, screen 11A, and the line 36A on screen 11A correspond to the iPhone 10, display screen 11, and line 36 that are shown in FIG. 3 and that the user will employ to measure the width of his erect penis. Accordingly, the display of FIG. 10 illustrates how a user positions an iPhone 10A (i.e., the user's iPhone 10) laterally, face-up, over the base of his erect penis with line 36A (i.e., line 36 on the user's iPhone) over and in registration with one side 43 of the penis.

Tapping the “BACK” button would sequence the program back to the primary instruction display shown in FIG. 3. Instead, the user taps the “NEXT” button 47 to sequence to the next supplemental instruction display illustrated in FIG. 11.

The display illustrated in FIG. 11 is identical to that shown in FIG. 10 except that (1) dashed lines depict the sides of penis 40 beneath iPhone 10A, and (2) the tip of the index finger 51 is shown tapping screen 11A at a point directly above side 44 of penis 40. Tapping screen 11A above side 44 determines the width of penis 40.

Tapping the “PREV” button 48 shown in FIG. 11 would sequence the program back to the supplemental instruction display shown in FIG. 10. Instead, the user taps the “BACK” button 46 shown in FIG. 11 to sequence back to the primary instruction display shown in FIG. 3.

The user follows the instructions set forth at the top of the display shown in FIG. 3 to measure the width of his erect penis. The user positions the iPhone 10 face-up laterally over the top of the base of his penis, aligns line 36 with one side 43 of the penis 40, and taps screen 11 at a point above the other side 44 of penis 40 to define the width of penis 40.

After the display of FIG. 3 is utilized to determine the width of penis 40, the user taps the “CONTINUE” button 22 (FIG. 3) to sequence to the next primary instruction display that is illustrated in FIG. 4.

### Height Determination

An align-touch measurement methodology is utilized to determine penile width. The iPhone 10 is placed in a “display screen 11 facing toward one side” third orientation generally parallel to and facing one side of an erect penis. In the third orientation, iPhone 10 is generally rotated ninety degrees about its longitudinal axis and from the first orientation. This third orientation is illustrated in FIGS. 12 and 13.

The primary instruction display illustrated in FIG. 4 includes “SHOW ME HOW” button 26, “CONTINUE” button 27, and “BACK” button 28.

The primary instruction display of FIG. 4 also includes the words “BOTTOM OF PENIS” 24 in conjunction with an arrow that is pointing to the side 56, indicating that side 56 is to be aligned with the bottom 55 (FIG. 12) of the user's erect penis.

The display of FIG. 4 includes at the top of the display an instruction paragraph which begins “Hold the phone . . . .” After reading the instruction paragraph, the user taps the “SHOW ME HOW” button 26 to index the program to the supplemental instruction display shown in FIG. 12. This supplemental instruction display depicts a side view of penis 40 including top 42, sides 43 and 44, and bottom 55. The display also depicts an iPhone 10A with a display screen 11A having a side 56A. The iPhone 10A, screen 11A, and side 56A correspond to the iPhone 10, display screen 11, and side 56 that are shown in FIG. 4 and that the user will employ to measure the height of his erect penis. Accordingly, the display of FIG. 12 illustrates how a user positions an iPhone 10A (i.e., the user's iPhone 10) on edge, facing the side 44 of his erect penis with the edge 56A (i.e., the edge 56 of the display screen 11 of the user's phone 10) adjacent and in registration with the bottom 55 of the user's penis 40.

Tapping the “BACK” button 46 in the display of FIG. 12 would sequence the program back to the primary instruction display shown in FIG. 4. Instead, the user taps the “NEXT” button 47 in FIG. 12 to sequence to the next supplemental instruction display illustrated in FIG. 13.

The display illustrated in FIG. 13 is identical to that shown in FIG. 12 except that the tip of the index finger 57 of hand 56 is shown tapping screen 11A at a point directly above the top 42 of penis 40. Tapping screen 11A at a point above top 42 determines the height of penis 40.

Tapping the “PREV” button 48 shown in FIG. 13 would sequence the program back to the supplemental instruction display shown in FIG. 12. Instead, the user taps the “BACK” button 46 shown in FIG. 13 to sequence back to the primary instruction display shown in FIG. 4.

The user follows the instructions set forth at the top of the display shown in FIG. 4 to measure the height of his erect penis. The user positions the iPhone 10 on edge, facing in, against one side of penis 40 with side 56 of screen 11 adjacent and aligned with the bottom 55 of penis 40. The user then taps screen 11 at a point adjacent and in alignment with the top 42 of penis 40 to define the height of penis 40.

After the display of FIG. 4 is utilized to determine the height of penis 40, the user taps the “CONTINUE” button 27 (FIG. 4) to sequence to the next primary instruction display that is illustrated in FIG. 5

The primary instruction display of FIG. 5 depicts the custom condom size E33, indicated by reference character 31, that has been calculated based on the length, width, and height measurements made by the user by employing the displays illustrated in FIGS. 2, 3, and 4. The primary instruction display shown in FIG. 5 also indicates the custom condom can be ordered at www.theyfit.com. If the user taps the “RETRY?” button, the program sequences back to the display shown in FIG. 2 to begin anew the measurement process.

### Girth Calculation

A particular problem encountered during the development of the invention concerns determination of the girth of an erect penis. A reasonably accurate determination of the girth of an erect penis is important in defining the size of condom which closely fits the penis. Even though the height and width of an erect penis may at first blush appear similar, or even equivalent, the circumference of the cross section of an erect penis is not circular; nor is it elliptical. However, experimentation during development of the invention determined that the cross section of an erect penis 40 is more nearly ovate and better approximated by an ellipse. Portions of the circumference of a penile cross-section can be concave while other portions are convex. There are various formulas for determining the perimeter of an ellipse based on the length of the axes a and b of the ellipse. In general, as the formulas become more complicated, they tend to more accurately estimate the perimeter of a given ellipse. Since, however, the cross section of an erect penis is not a perfect ellipse, it is believed that focusing on closely calculating the perimeter (and therefore the girth) of an ellipse with axes comprising the width and height of an erect penis 40 is not a desirable goal. The goal is to develop an algorithm which provides an acceptably accurate approximation of the girth of an erect penis. One approach to developing an acceptable algorithm is to carefully measure the dimensions of a selected number of different erect penises and mathematically derive a specialized algorithm to calculate girth. Other approaches may be utilized. For example, the formula for determining the perimeter of a circle, or hexagon, or pentagon, etc. can be employed, possibly by incorporating a variable into the formula which would compensate for any inherent error in the formula. At present it has been determined that the algorithm:

C=(2x)^{1/2} (1)

where x=(a/2)²+(b/2)² (2)

provides a close approximation of the girth of an erect penis, where a equals the width, b equals the height of the erect penis, and C equals the approximated circumference, or girth, of an erect penis 40.

### Introducing Snugness Factor

Once the value of the girth of a penis 40 is calculated using the algorithm (1) noted above, a condom with an appropriate snugness factor is selected along with the measured length of the erect penis 40 to determine the appropriate condom size from a table that sets forth condom lengths in combination with condom circumferences calculated use. The lay flat width is the width of a condom when it is lying flat on a table top or other surface; consequently, the outer circumference of a condom is about twice the “lay flat width”.

Providing a condom with a calculated lay circumference (i.e., where the “calculated lay circumference” equals twice the lay flat width) that is equivalent to the girth of an erect penis does not provide a condom with an acceptable fit. The condom will only loosely fit because the condom is not elastically stretched an amount sufficient to generate frictional forces which make it unlikely that the condom will come off during sexual activity. If a condom, when placed on an erect penis, is not stretched radially, or is only stretched a small amount, it is likely the condom will inadvertently come off during sexual activity. The proportional amount by which the girth of an erect penis must exceed the calculated lay circumference of a condom is herein deemed the snugness factor, and is expressed as a percentage of the calculated lay circumference of the condom. During development of the invention, it was determined that as the size of a condom increases, the snugness factor does not remain the same but, instead, gradually increases. For example, a condom with a calculated lay circumference of 82 mm can have a snugness factor of 12%, which means the condom should be used on an erect penis having a girth equal to about 82+(0.12×82)=92 mm. And, a condom with a calculated lay circumference of 138 mm can have a snugness factor of 31.3%, which means the condom should be used on an erect penis having a girth that is 31.3% greater than the calculated lay circumference and that is equal to about 138+(0.313×138)=171 mm. The appropriate value of the snugness factor for a condom with a particular calculated lay circumference may vary depending on the chemical processing of the latex used to produce the condom by a manufacturer. The snugness factor is readily determined by testing various condom sizes on erect penises having known calculated girths.

By way of example, and limitation, TABLE I below illustrates the range of penile girths necessary for particular sizes of a brand of latex condoms, along with the concomitant snugness factor range:

TABLE I
Increased Penile Girth Necessary for Proper Fit of Condom

| | | Range of | Range of |
|---|---|---|---|
| | Calculated Lay | Acceptable | Acceptable |
| Condom | Circumference | Penile Girths | Snugness Factors |
| Model | (mm) | (mm) | (% increase) |
| 33 | 82 | 89 to 95 | 9 to 16 |
| 44 | 86 | 97 to 102 | 13 to 19 |
| 55 | 90 | 103 to 109 | 14 to 21 |
| 66 | 94 | 110 to 117 | 17 to 24 |
| 77 | 98 | 118 to 123 | 20 to 25 |
| 88 | 102 | 124 to 129 | 22 to 26 |
| 99 | 106 | 130 to 135 | 23 to 27 |
| 11 | 110 | 136 to 141 | 24 to 28 |
| 17 | 114 | 142 to 150 | 25 to 32 |
| 21 | 120 | 151 to 161 | 26 to 34 |
| 22 | 128 | 162 to 175 | 27 to 35 |
| 31 | 138 | 176+ | 28+ |

In use of the above table, when the girth of an erect penis is calculated, it is rounded off to the nearest whole number. I.e., if the girth of an erect penis is calculated at 89.4, this is rounded off to 89 and a Condom Model 33 with a lay circumference is selected. Once the proper condom model for a particular measured penile girth is selected, the length of the erect penis is utilized to select the proper version of the model. For example C can indicate a penile length in the range of 75 mm to 100 mm, B can indicate a penile length in the range of 50 mm to 75 mm, E can indicate a penile length in the range of 101 to 125 mm, F can indicate a length in the range of 126 mm to 150 mm, G can indicate a length in the range of 151 to 175 mm, H an indicate a length in the range of 176 to 200 mm, and J can indicate a length greater than 200 mm. Consequently, if the penile girth is 106 mm and the length is 110 mm, the condom selected would be a 55E, where Model 55 is selected from Table I above and the “E” indicates a penile length in the range of 101 to 125 mm, as noted.

An iTunes or other computer application that is downloaded onto a mobile computer device to operate in accordance with the invention could include the entire Table I above, but would, as would be appreciated by those of skill in the art, really only require the information in the first column (“Condom Model”) and the third column (Range of Acceptable Penile Girths). Further, how the computer application determines the proper condom model or size to select can vary as desired as long as the condom selected has a calculated lay circumference which is less than the girth of the erect penis, such that the girth is greater than the calculated lay circumference.

The block diagram of FIG. 14 illustrates at least in part a computer system incorporated into iPhone 10. A computer in iPhone 10 includes controller 60 and memory 61. Controller 60 includes control 63 and sub-routines 64 utilized in a computer program constructed in accordance with the invention. Data input 65 can comprise commands entered in iPhone 10 by tapping the display screen 11, can comprise computer programs downloaded into iPhone 10, or can comprise data entered in iPhone 10 by any other desired means. Displays on screen 11 are produced in response to commands generated by controller 60.

The block diagram of FIG. 15 depicts sub-routines 64 employed in a computer program constructed in accordance with the invention, and depicts data stored in memory 61. The sub-routines 64 can include promotional sub-routine 80 which utilizes promotional data 81 to produce promotional material on an opening display on screen 11. An example of such promotional material is described above as appearing in the initial display which appears on screen 11 prior to the display of FIG. 1.

The right/left hand sub-routine 66 operates in conjunction with right/left hand data 67 and with the display of FIG. 1 to allow a user to identify whether he right or left handed. Whether a user is right handed or left handed affects the displays that are produced on screen 11. For example, if a user indicates that he is left handed, then in the display of FIG. 2, tape 31 appears along the left hand side of screen 11 in the manner depicted in FIG. 2. If a user indicates that he is right handed, then in the display of FIG. 2 the tape 31 would appear along the right hand side of screen 11.

The length measurement sub-routine 68 operates in conjunction with the display of FIG. 2 and with length data 69 generated during use of the display of FIG. 2 to determine the length of the user's erect penis.

The width measurement sub-routine 70 operates in conjunction with the display of FIG. 3 and with width data 71 generated during use of the display of FIG. 3 to determine the width of the user's erect penis.

The height measurement sub-routine 72 operates in conjunction with the display of FIG. 4 and with height data 73 generated during use of the display of FIG. 4 to determine the height of the user's erect penis.

The correlation sub-routine 74 calculates the girth measurement of the user's erect penis and then compares the girth measurement and length measurements to condom size data which is in the form of a specialized table which identifies various condoms as each being appropriate for a length measurement in a particular range and a girth measurement in a particular range. The linear units of measure utilized in the table and in the length and girth measurements can be from the metric (i.e., centimeters, millimeters, etc.) or English (i.e., inches) systems of measurement. For example, an E33 condom can be appropriate for a penile girth measurement in the range of 89 to 95 mm and a penile length measurement in the range of 101 to 125 mm. If the length and girth measurements determined for a particular user via FIGS. 2, 3, and 4 are 110 mm and 90 mm, then the correlation sub-routine selects the E33 condom and this customized condom appears as shown in the display of FIG. 5 and identified by reference character 31.

The ordering sub-routine 76 operates in conjunction with ordering data 77 and with the display of FIG. 5 to connect a user to the www.theyfit.com web site on the Internet to order a customized condom(s).

The navigation sub-routine 78 operates in conjunction with navigation data 79 and with the displays of FIGS. 1 to 13 to permit a user to navigate from display to display by tapping the “BACK”, “PREV”, “CONTINUE”, etc. buttons.

FIGS. 16 to 21 set forth, by way of example and not limitation, an example of a methodology which can be used according to the principles of the invention.

In FIG. 16, the first step 84 is to prepare an iTunes application for selecting a customized condom based on length, width, and height measurements obtained using the application in combination with an iPod, iPhone, etc. The application can operate in the manner described earlier herein with respect to FIGS. 1 to 13, or can function in any other desired manner to obtain length and girth measurements for an erect penis. The application need not, of course, be an iTunes application but can be any application formatted for use on any desired portable electronic digital computer device.

The second step 85 comprises publishing the iTunes application for sale in iTune on the iTune on-line store.

In step 86, the iTunes application is sold and is downloaded to an iPhone, or other desired portable electronic digital computer device.

In step 87, the iTunes application is opened on the iPhone.

In step 88, when the iTunes application is opened on the iPhone, the promotional display is viewed and promotional material on the promotional display is read. An example of such promotional material is described earlier herein.

In step 89, a “CONTINUE” button on the promotional page is tapped and the left/right hand display of FIG. 1 appears. The “LEFT HANDED” 12 or “RIGHT HANDED” 13 button, as appropriate, is tapped to continue on to the length measurement display of FIG. 2, which is a primary instruction display.

In step 90 (FIG. 17), the “SHOW ME HOW” button in the display of FIG. 2 is tapped to go to the first supplemental instruction display illustrated in FIG. 6.

In step 91, the first supplemental instruction display is read, and the “NEXT” button 47 in FIG. 6 is tapped to go to the second supplemental instruction display illustrated in FIG. 7.

In step 92, the second supplemental instruction display is read and the “NEXT” button 47 in FIG. 7 is tapped to go to the third supplemental instruction display illustrated in FIG. 8.

In step 93, the third supplemental instruction display is read and the “BACK” button 46 in FIG. 8 is tapped to return to the length measurement display of FIG. 2.

In step 94 the iPhone is placed adjacent one side of an erect penis 40 as illustrated in FIG. 6, with the bottom edge 35 of the screen 11 adjacent the base 45 of the penis 40 and the iPhone 10 extending along the side 43 of and parallel to the penis 40.

In step 95, the index finger 51 of a hand 50 is placed against screen 11 on one of the boxes 34 on tape 31 to “TACK DOWN” tape 31. The hand 50 and finger 51 are held in fixed position.

In step 96, the user's other hand is used to moved iPhone 10 along penis 40 in the direction of arrows A (FIGS. 7 and 8) to scroll along tape 31. Moving iPhone 10 in the direct of arrows A moves iPhone 10 away from the user's body.

In step 97, after the iPhone 10 has bee moved far enough in the direction of arrow A so that one of the boxes 52 on tape 31 is adjacent the tip 41 of penis in the manner illustrated in FIG. 9, the iPhone is held in fixed position with box 52 adjacent tip 41, finger 51 is moved up off display screen 11, is moved to a position over box 52, and is used to tap box 52. This defines the length of penis 40.

In step 98, the user taps the “CONTINUE” button 17 in FIG. 2 to continue to the width measurement display illustrated in FIG. 3. The display of FIG. 3 is a primary instruction display.

In step 99, the “SHOW ME HOW” button 21 in FIG. 3 is tapped to go to the fourth supplemental instruction display illustrated in FIG. 10.

In step 100, the fourth supplemental instruction display of FIG. 10 is read and the “NEXT” button 47 in FIG. 10 is tapped to go to the fifth supplemental instruction display illustrated in FIG. 11.

In step 101, the fifth supplemental instruction display of FIG. 11 is read and the “BACK” button in FIG. 11 is tapped to return to the width measurement display of FIG. 3.

In step 102, the iPhone is, as illustrated in FIG. 10, placed face up in a horizontally oriented configuration on top of and generally perpendicular to penis 40 and with line 36 positioned above and in registration with one side 43 of penis 40.

In step 103, display screen 11 is tapped at a point directly above and in registration with the other side 44 of penis 40. This defines the width of penis 40.

In step 104, the “CONTINUE” button 22 in FIG. 3 is tapped to continue to the height measurement display illustrated in FIG. 4. The display of FIG. 4 is a primary instruction display.

In step 105, the “SHOW ME HOW” button 26 is tapped to go to the sixth supplemental instruction display of FIG. 12.

In step 106 the sixth supplemental instruction display of FIG. 12 is read and the “NEXT” button 47 in FIG. 12 is tapped to go to the seventh supplemental instruction display of FIG. 13.

In step 107, the seventh supplemental instruction display of FIG. 13 is read and the “BACK” button 46 in FIG. 13 is tapped to return to the height measurement display of FIG. 4.

In step 108, the iPhone is, as illustrated in FIG. 12, held on edge in a vertically oriented configuration with (1) display 11 facing and against one side of penis 40 and (2) the left edge 56 of display 11 adjacent and in registration with the bottom 55 of penis 40.

In step 109, screen 11 is tapped at a point adjacent and in registration with the top 42 of penis 40. This defines the height of penis 40.

In step 110, the “CONTINUE” button 27 in FIG. 4 is tapped to continue to the ordering display of FIG. 5.

In step 111, the screen 11 is tapped above www.theyfit.com to connect to a web site to order a custom fitted condom size E33.

In an alternate embodiment of the invention, the hand held computer device is provide with a first specialized computer program which determines the length, height, and width of an erect penis by comparing proportionally the penis to an object having a known dimension. The hand held computer device includes a camera. The camera views, or takes a picture of, an erect penis with an object of known length, for instance a credit card, adjacent the penis. The specialized computer program knows the length of the credit card. The specialized computer program proportionately compares the length (or width or height) of the penis to the credit card and then makes the necessary calculation. For example, if the length of the penis is twice that of the credit card, and the length of the credit card is two inches, then the specialized computer program computes the length of the penis as four inches. The height and width of the penis are utilized in formula (1) above, or in another selected or derived algorithm, to determine the girth of the penis. The length and girth measurements are utilized to select a customized condom based on a table or other data stored in the first specialized computer program.

In still another embodiment of the invention, the hand held computer is constructed to include a roller or optical sensor or other sensor and to include a second specialized computer program. When the computer is placed on a surface and is moved a selected distance, the roller or sensor enable the second specialized computer program to calculate the distance that the handheld computer is moved. Consequently, for example, the handheld computer can be positioned adjacent the penis with the roller contacting the base of an erect penis, the touch display screen of the hand held computer can be tapped, the handheld computer can be moved so the roller rolls along the penis to the distal tip of the penis, and the touch display screen of the handheld computer tapped a second time to define the length of the penis. When the length is determined, the spatial position or location of the cell phone changes as the roller moves along the length of the penis. The girth of the penis can be determined in a similar manner by manipulating the handheld computer to move the roller completely around the penis. When the girth is determined, the spatial position or location of the cell phone constantly changes while the roller moves over the penile circumference. The length and girth measurements are utilized to select a customized condom based on a table or other data stored in the second specialized computer program.

In yet another embodiment of the invention, an accelerometer is incorporated in a handheld computer along with a third specialized computer program. The handheld computer is held stationary at the base of an erect penis, the display screen 11 is tapped, the handheld computer (and accelerometer therein) is moved to the tip of the penis, and the display screen 11 is tapped a second time. The third specialized computer program utilizes signals generated by the accelerometer to calculate the length of the penis. When the length is determined in this manner, the spatial position or location of the handheld computer changes as the computer is moved along the length of the penis. The girth of the penis is determined in a similar manner. When the girth of the erect penis is determined, the spatial position or location of the handheld computer changes as the handheld computer is moved around the circumference of the penis. The length and girth measurements are utilized to select a customized condom based on a table or other data stored in the third specialized computer program.

A specialized computer program that is utilized in conjunction with a handheld computer like an iPod, iPhone, etc. can enable an individual to select further customizing features such as, by way of example and not limitation, the kind of lubricant, a preferred texture (dots, ribs, smooth, etc.), the kind of material used to make a condom (latex, polyurethane, etc.), and shape (generally cylindrical cross section, flared, etc.). Such selections can also be provided at a web site, for example www.theyfit.com, at which an individual orders a condom.

In another embodiment of the invention, a first party pays a second party retailer that markets customized condoms and that markets a specialized computer program to measure an erect penis and select a customized condom that will provide a good fit. The payment is, however, made on behalf of a third party on the condition that the anonymity of the first party is maintained. The second party retailer then notifies the third party by text message, by e-mail, or by another selected communication system that payment has been received from an anonymous source by the second party retailer so that the third party can visit an Internet web site and download off the necessary specialized computer program for a handheld computer for measuring an erect penis and selecting an appropriately sized condom and/or purchase a selected number of condoms via the Internet web site. The third party then has the option of taking advantage of such pre-paid services. If the third party takes advantage of the pre-paid services and products, the second party retailer notifies the first party that their pre-paid services or products were claimed.

In another embodiment of the invention the handheld computer or other device utilized by an individual uses image recognition software which can, when an optical system or other selected sensing system in the handheld computer “views” an erect penis, take into account the distance of the handheld computer from the erect penis and can accurately and automatically calculate the dimensions (i.e., length, width, height, and/or girth) of the erect penis by appropriately positioning the handheld computer with respect to the erect penis. It is anticipated that the handheld computer will, in order to accurately determine the dimensions of an erect penis, have to be placed in at least two separate positions (for example on top of and then at one side of the penis) with respect to the penis or have two or more cameras.

Having described the invention and presently preferred embodiments and the best modes thereof in such terms as to enable one of skill in the art to make and use the invention, I claim:

1. A method for fitting a wearable covering for a body part, comprising the steps of:
a) running a program on a portable terminal;
b) determining with the portable terminal specific geometrical points regarding the body part;
c) using the determined specific geometrical points to determine an approximate shape of the body part;
d) comparing the determined approximate shape with a table of different sized wearable coverings for the body part; and
e) selecting a size for the wearable covering.

2. A method for an individual to obtain a condom, comprising the steps of
(a) designing a computer program for use on a hand held computer device, said device including a touch sensitive display screen on which data commands are entered by touching said display screen at selected areas on said display screen, said program utilizing
(i) said touch screen to measure the length, width, and height of an erect penis,
(ii) a specialized algorithm to calculate the girth of an erect penis based on the height and width of the penis, the penis including a top, a bottom, and a pair of spaced apart generally parallel sides, and
(iii) a chart correlating the length and girth of an erect penis to a particular size of condom,
said hand held computer device being
(iv) held in fixed position adjacent an erect penis during measurement of each dimension in a group of dimensions consisting of the length, the width, and the height of an erect penis, and
(v) held, during measurement of the length of an erect penis, initially in fixed position adjacent the erect penis and then being moved to scroll along a measuring tape depicted on said display screen of said computer device;
(b) offering said computer program for sale on the Internet;
(c) selling said computer program on the Internet;
(d) downloading said computer program over the Internet to said handheld computer device;
(e) utilizing said handheld computer device and computer program to measure the length of an erect penis of an individual by initially placing said computer device adjacent the erect penis and then moving said computer device along the erect penis away from the body of the individual to scroll along a measuring tape depicted on said display screen of said computer device;
(f) utilizing said handheld computer device and computer program to measure the width of the individual's erect penis while holding said computer device in fixed position on the top or bottom of the penis;
(g) utilizing said handheld computer device and computer program to measure the height of the individual's erect penis while holding said computer device in fixed position along one of the sides of the penis;
(h) utilizing said width, height, and algorithm to determine the girth of the penis;
(i) utilizing said length and girth to select a particular size of condom from said chart;
(j) utilizing an Internet web site to order said particular size of condom.
